(19) United States
(12) Patent Application Publication
(10) Pub. No.: US 2011/0097779 A1
(43) Pub. Date: Apr. 28, 2011

(54) Processes for Producing Fermentation Products

(76) Inventors: Chee-Leong Soong, Raleigh, NC (US); Peter Rahbek, Virum (DK); Shiro Fukuyama, Chiba (JP); Jiyin Liu, Raleigh, NC (US); Randy Deinhammer, Wake Forest, NC (US); Martin Simon Borchert, Alleroed (DK); Suzanne Clark, Wake Forest, NC (US)

(21) Appl. No.: 12/993,522

(22) PCT Filed: Jun. 23, 2009
(86) PCT No.: PCT/US09/48286

§ 371 Date: Nov. 19, 2010

Related U.S. Application Data
(60) Provisional application No. 61/074,762, filed on Jun. 23, 2008.

(51) Int. Cl. C12P 7/06; C12N 9/26

(52) U.S. Cl. 435/161; 435/201

(57) ABSTRACT

The present invention relates to processes for producing a fermentation product from gelatinized and/or un-gelatinized starch-containing material using a metallo protease, and processes for producing a fermentation product from gelatinized starch-containing material using a metallo protease and a pullulanase.

### FIELD OF THE INVENTION

The present invention relates to processes for producing fermentation products from gelatinized and/or un-gelatinized starch-containing material.

### BACKGROUND OF THE INVENTION

Production of fermentation products, such as ethanol, from starch-containing material is well-known in the art. Generally two different kinds of processes are used. The most commonly used process, often referred to as a “conventional process”, includes liquefying gelatinized starch at high temperature using typically a bacterial alpha-amylase, followed by simultaneous saccharification and fermentation carried out in the presence of a glucoamylase and a fermentation organism. Another well known process, often referred to as a “raw starch hydrolysis”-process (RSH process) includes simultaneously saccharifying and fermenting granular starch below the initial gelatinization temperature typically in the presence of an acid fungal alpha-amylase and a glucoamylase.

U.S. Pat. No. 5,231,017-A discloses the use of an acid fungal protease during ethanol fermentation in a process comprising liquefying gelatinized starch with an alpha-amylase.

WO 2003/066826 discloses a raw starch hydrolysis process (RSH process) carried out on non-cooked mash in the presence of fungal glucoamylase, alpha-amylase and fungal protease.

WO 2007/145912 discloses a process for producing ethanol comprising contacting a slurry comprising granular starch obtained from plant material with an alpha-amylase capable of solubilizing granular starch at a pH of 3.5 to 7.0 and at a temperature below the starch gelatinization temperature for a period of 5 minutes to 24 hours; obtaining a substrate comprising greater than 20% glucose, and fermenting the substrate in the presence of a fermenting organism and starch hydrolyzing enzymes at a temperature between 10° C. and 40° C. for a period of 10 hours to 250 hours. Additional enzymes added during the contacting step may include protease.

WO 2006/028897 discloses a process for liquefying starch-containing material comprising treating alpha-amylase treated starch with a pullulanase at a temperature between 40° C. and 60° C. for a period of 20 to 90 minutes.

There is still a desire and need for providing improved processes for producing fermentation products, such as ethanol, from starch-containing material.

### SUMMARY OF THE INVENTION

The present invention relates to processes of producing fermentation products, such as ethanol, from gelatinized as well as un-gelatinized starch-containing material using a fermenting organism.

In the first aspect the invention relates to processes for producing fermentation products from starch-containing material comprising simultaneously saccharifying and fermenting starch-containing material using a carbohydrate-source generating enzyme and a fermenting organism at a temperature below the initial gelatinization temperature of said starch-containing material in the presence of a metallo protease.

In a second aspect the invention relates to processes for producing fermentation products from starch-containing material comprising the steps of:

(a) liquefying starch-containing material in the presence of an alpha-amylase;

(b) saccharifying the liquefied material obtained in step (a) using a carbohydrate-source generating enzyme;

(c) fermenting using a fermenting organism;

wherein a metallo protease is present i) during fermentation, and/or ii) before, during, and/or after liquefaction.

In a third aspect the invention relates to processes for producing fermentation products from starch-containing material comprising the steps of:

(a) liquefying starch-containing material in the presence of an alpha-amylase;

(b) saccharifying the liquefied material obtained in step (a) using a carbohydrate-source generating enzyme;

(c) fermenting using a fermenting organism;

wherein a metallo protease is present i) during fermentation, and/or ii) before, during, and/or after liquefaction, and a pullulanase is present i) during fermentation, and/or ii) before, during, and/or after liquefaction.

The invention also relates to composition comprising a metallo protease, a carbohydrate-source generating enzyme, and an alpha-amylase, and a composition comprising a metallo protease and a pullulanase, and/or a carbohydrate-source generating enzyme and/or an alpha-amylase. Finally the invention relates to the use of metallo protease in a process for fermenting gelatinized and/or un-gelatinized starch-containing material into a fermentation product, or the use of metallo protease and pullulanase in a process for fermenting gelatinized starch-containing material into a fermentation product.

### DETAILED DESCRIPTION OF THE INVENTION

The present invention relates to processes of producing fermentation products, such as ethanol, from gelatinized as well as un-gelatinized starch-containing material using a fermenting organism.

The inventors have found that when using a metallo protease derived from Thermoascus aurantiacus CGMCC No. 0670 or a metalloprotease derived from Aspergillus oryzae in a raw starch hydrolysis process (RSH process), the fermentation rate was boosted and the ethanol yield increased compared to when not adding a metallo protease or when adding a protease selected from other protease groups, to a corresponding process. Further, the inventors found that when adding a metallo protease derived from Thermoascus aurantiacus CGMCC No. 0670 to a conventional ethanol process, the ethanol yield was improved. Surprisingly, the addition of both the metallo protease and a thermostable pullulanase from Pyrococcus woesei to a conventional ethanol process boosted the ethanol yield more than either the metallo protease or pullulanase alone, suggesting a synergistic effect on ethanol yield.

### Metallo Proteases

The term “protease” as used herein is defined as an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see, e.g., the World Wide Web (WWW) at www.chem.qmw.ac.uk/iubmb/enzyme/index.html.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

The term “metallo protease” as used herein is defined as a protease selected from the group consisting of:
(a) proteases belonging to EC 3.4.24 (metalloendopeptidases); preferably EC 3.4.24.39 (acid metallo proteinases);
(b) metallo proteases belonging to the M group of the above Handbook;
(c) metallo proteases not yet assigned to clans (designation: Clan MX), or belonging to either one of clans MA, MB, MC, MD, ME, MF, MG, MH (as defined at pp. 989-991 of the above Handbook);
(d) other families of metalloproteases (as defined at pp. 1448-1452 of the above Handbook);
(e) metallo proteases with a HEXXH motif;
(f) metallo proteases with an HEFTH motif;
(g) metallo proteases belonging to either one of families M3, M26, M27, M32, M34, M35, M36, M41, M43, or M47 (as defined at pp. 1448-1452 of the above Handbook);
(h) metalloproteases belonging to the M28E family; and
(i) metalloproteases belonging to family M35 (as defined at pp. 1492-1495 of the above Handbook).

In other particular embodiments, metallo proteases are hydrolases in which the nucleophilic attack on a peptide bond is mediated by a water molecule, the water molecule being activated by a divalent metal cation. Examples of divalent cations are zinc, cobalt or manganese. The metal ion may be held in place by amino acid ligands. The number of ligands may be five, four, three, two, one or zero. In a particular embodiment the number is two or three, preferably three.

For determining whether a given protease is a metallo protease or not, reference is made to the above Handbook and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein). Two protease assays are described below in the “Materials & Methods”-section, of which the so-called AZCL-Casein Assay is the preferred assay.

There are no limitations on the origin of the metallo protease used in a process of the invention. In an embodiment the metallo protease is classified as EC 3.4.24, preferably EC 3.4.24.39. In one embodiment, the metallo protease used according to the invention is an acid-stable metallo protease, more preferable a fungal acid-stable metallo protease, such as a metallo protease derived from a strain of the genus Thermoascus, preferably a strain of Thermoascus aurantiacus, especially Thermoascus aurantiacus CGMCC No. 0670 (classified as EC 3.4.24.39). In another embodiment, the metallo protease is derived from a strain of the genus Aspergillus, preferably a strain of Aspergillus oryzae.

The metallo proteases include not only natural or wild-type metallo proteases, but also any mutants, variants, fragments etc. thereof exhibiting metallo protease activity, as well as synthetic metallo proteases, such as shuffled metallo proteases, and consensus metallo proteases. Genetically engineered metallo proteases can be prepared as is generally known in the art, e.g., by Site-directed Mutagenesis, by PCR (using a PCR fragment containing the desired mutation as one of the primers in the PCR reactions), or by Random Mutagenesis. The preparation of consensus proteins is described in, e.g., EP 897,985. The term “obtained from” as used herein in connection with a given source shall mean that the polypeptide encoded by the nucleic acid sequence is produced by the source or by a cell in which the nucleic acid sequence from the source is present. In a preferred embodiment, the polypeptide is secreted extracellularly.

In one embodiment the metallo protease is an isolated polypeptide comprising an amino acid sequence which has a degree of identity to amino acids −178 to 177, −159 to 177, or preferably amino acids 1 to 177 (the mature polypeptide) of SEQ ID NO:1 herein of at least about 80%, or at least about 82%, or at least about 85%, or at least about 90%, or at least about 95%, or at least about 97%; and which have metallo protease activity (hereinafter “homologous polypeptides”). In particular embodiments, the metallo protease consists of an amino acid sequence with a degree of identity to SEQ ID NO: 1 as mentioned above.

The Thermoascus aurantiacus metallo protease, the mature polypeptide of which comprises amino acids 1-177 of SEQ ID NO: 1 herein is a preferred example of a metallo protease suitable for use in a process of the invention. Another homologous polypeptide is derived from Aspergillus oryzae and comprises SEQ ID NO: 3 herein (and SEQ ID NO: 11 disclosed in WO 2003/048353), or amino acids −23-353; −23-374; −23-397; 1-353; 1-374; 1-397; 177-353; 177-374; or 177-397 thereof, and is encoded by SEQ ID NO: 2 herein and SEQ ID NO: 10 disclosed in WO 2003/048353.

Another metallo protease suitable for use in the process of the invention is the Aspergillus oryzae metallo protease comprising SEQ ID NO: 5 herein. In one embodiment the metallo protease is an isolated polypeptide comprising an amino acid sequence which has a degree of identity to SEQ ID NO: 5 herein of at least about 80%, or at least about 82%, or at least about 85%, or at least about 90%, or at least about 95%, or at least about 97%; and which have metallo protease activity (hereinafter “homologous polypeptides”). In particular embodiments, the metallo protease consists of an amino acid sequence with a degree of identity to SEQ ID NO: 5 as mentioned above.

In a particular embodiment, a homologous polypeptide has an amino acid sequence that differs by forty, thirtyfive, thirty, twentyfive, twenty, or by fifteen amino acids from amino acids −178 to 177, −159 to 177, or +1 to 177 of SEQ ID NO: 1 herein or from SEQ ID NO: 5 herein.

In another embodiment, a homologous polypeptide has an amino acid sequence that differs by ten, or by nine, or by eight, or by seven, or by six, or by five amino acids from amino acids −178 to 177, −159 to 177, or +1 to 177 of SEQ ID NO: 1 herein or SEQ ID NO: 5 herein. In another particular embodiment, a homologous polypeptide differ by four, or by three, or by two amino acids, or by one amino acid from amino acids −178 to 177, −159 to 177, or +1 to 177 of SEQ ID NO: 1 herein or SEQ ID NO: 5 herein.

In particular embodiments, the metallo protease a) comprise, or b) consist of

i) the amino acid sequence of amino acids −178 to 177, −159 to 177, or +1 to 177 of SEQ ID NO:1 herein;

ii) the amino acid sequence of amino acids −23-353, −23-374, −23-397, 1-353, 1-374, 1-397, 177-353, 177-374, or 177-397 of SEQ ID NO: 3 herein;

iii) the amino acid sequence of SEQ ID NO: 5 herein; or allelic variants, or fragments, of the sequences of i), ii), and iii) that have protease activity.

A fragment of amino acids −178 to 177, −159 to 177, or +1 to 177 of SEQ ID NO: 1 herein or of amino acids −23-353, −23-374, −23-397, 1-353, 1-374, 1-397, 177-353, 177-374, or 177-397 of SEQ ID NO: 3 herein; is a polypeptide having one or more amino acids deleted from the amino and/or carboxyl terminus of these amino acid sequences. In one embodiment a fragment contains at least 75 amino acid residues, or at least 100 amino acid residues, or at least 125 amino acid residues, or at least 150 amino acid residues, or at least 160 amino acid residues, or at least 165 amino acid residues, or at least 170 amino acid residues, or at least 175 amino acid residues.

An allelic variant denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

In another embodiment the metallo protease is combined with other proteases, such as fungal proteases, preferably acid fungal proteases.

Processes for Producing Fermentation Products from Un-Gelatinized Starch-Containing Material

In this aspect the invention relates to processes for producing fermentation products from starch-containing material without gelatinization (i.e., without cooking) of the starch-containing material. According to the invention the desired fermentation product, such as ethanol, can be produced without liquefying the aqueous slurry containing the starch-containing material and water. In one embodiment a process of the invention includes saccharifying (e.g., milled) starch-containing material, e.g., granular starch, below the initial gelatinization temperature, preferably in the presence of alpha-amylase and/or carbohydrate-source generating enzyme(s) to produce sugars that can be fermented into the desired fermentation product by a suitable fermenting organism.

In this embodiment the desired fermentation product, preferably ethanol, is produced from un-gelatinized (i.e., uncooked), preferably milled, cereal grains, such as corn.

Accordingly, in the first aspect the invention relates to processes for producing fermentation products from starch-containing material comprising simultaneously saccharifying and fermenting starch-containing material using a carbohydrate-source generating enzyme and a fermenting organism at a temperature below the initial gelatinization temperature of said starch-containing material in the presence of a metallo protease.

The fermentation product, such as especially ethanol, may optionally be recovered after fermentation, e.g., by distillation. Suitable starch-containing starting materials are listed in the “Starch-Containing Materials”-section below. Contemplated enzymes are listed in the “Enzymes”-section below. Typically amylase(s), such as glucoamylase(s) and/or other carbohydrate-source generating enzymes, and/or alpha-amylase(s), is(are) present during fermentation.

Examples of glucoamylases and other carbohydrate-source generating enzymes can be found below and includes raw starch hydrolysing glucoamylases.

Examples of alpha-amylase(s) include acid alpha-amylases, preferably acid fungal alpha-amylases.

Examples of fermenting organisms include yeast, preferably a strain of Saccharomyces cerevisiae. Other suitable fermenting organisms are listed in the “Fermenting Organisms”-section above.

The term “initial gelatinization temperature” means the lowest temperature at which starch gelatinization commences. In general, starch heated in water begins to gelatinize between about 50° C. and 75° C.; the exact temperature of gelatinization depends on the specific starch and can readily be determined by the skilled artisan. Thus, the initial gelatinization temperature may vary according to the plant species, to the particular variety of the plant species as well as with the growth conditions. In context of this invention the initial gelatinization temperature of a given starch-containing material may be determined as the temperature at which birefringence is lost in 5% of the starch granules using the method described by Gorinstein. S. and Lii. C., Starch/Stärke, Vol. 44 (12) pp. 461-466 (1992).

Before initiating the process a slurry of starch-containing material, such as granular starch, having 10-55 w/w-% dry solids (DS), preferably 25-45 w/w-% dry solids, more preferably 30-40 w/w-% dry solids of starch-containing material may be prepared. The slurry may include water and/or process waters, such as stillage (backset), scrubber water, evaporator condensate or distillate, side-stripper water from distillation, or process water from other fermentation product plants. Because the process of the invention is carried out below the initial gelatinization temperature, and thus no significant viscosity increase takes place, high levels of stillage may be used if desired. In an embodiment the aqueous slurry contains from about 1 to about 70 vol.-%, preferably 15-60% vol.-%, especially from about 30 to 50 vol.-% water and/or process waters, such as stillage (backset), scrubber water, evaporator condensate or distillate, side-stripper water from distillation, or process water from other fermentation product plants, or combinations thereof, or the like

The starch-containing material may be prepared by reducing the particle size, preferably by dry or wet milling, to 0.05 to 3.0 mm, preferably 0.1-0.5 mm. After being subjected to a process of the invention at least 85%, at least 86%, at least 87%, at least 88%, at least 89%, at least 90%, at least 91%, at least 92%, at least 93%, at least 94%, at least 95%, at least 96%, at least 97%, at least 98%, or preferably at least 99% of the dry solids in the starch-containing material are converted into a soluble starch hydrolysate.

A process in this aspect of the invention is conducted at a temperature below the initial gelatinization temperature, which means that the temperature typically lies in the range between 30-75° C., preferably between 45-60° C.

In a preferred embodiment the process carried at a temperature from 25° C. to 40° C., such as from 28° C. to 35° C., such as from 30° C. to 34° C., preferably around 32° C.

In an embodiment the process is carried out so that the sugar level, such as glucose level, is kept at a low level, such as below 6 w/w-%, such as below about 3 w/w-%, such as below about 2 w/w-%, such as below about 1 w/w-%., such as below about 0.5 w/w-%, or below 0.25 w/w-%, such as below about 0.1 w/w-%. Such low levels of sugar can be accomplished by simply employing adjusted quantities of enzyme and fermenting organism.

A skilled person in the art can easily determine which doses/quantities of enzyme and fermenting organism to use. The employed quantities of enzyme and fermenting organism may also be selected to maintain low concentrations of maltose in the fermentation broth. For instance, the maltose level may be kept below about 0.5 w/w-%, such as below about 0.2 w/w-%.

The process of the invention may be carried out at a pH from about 3 and 7, preferably from pH 3.5 to 6, or more preferably from pH 4 to 5. In an embodiment fermentation is ongoing for 6 to 120 hours, in particular 24 to 96 hours.

Processes for Producing Fermentation Products from Gelatinized Starch-Containing Material

In this aspect the invention relates to processes for producing fermentation products, especially ethanol, from starch-containing material, which process includes a liquefaction step and sequentially or simultaneously performed saccharification and fermentation steps.

Consequently, the invention relates to processes for producing fermentation products from starch-containing material comprising the steps of:

(a) liquefying starch-containing material in the presence of an alpha-amylase;

(b) saccharifying the liquefied material obtained in step (a) using a carbohydrate-source generating enzyme;

(c) fermenting using a fermenting organism;

wherein a metallo protease is present: i) during fermentation, and/or ii) before, during, and/or after liquefaction.

The invention also relates to processes for producing fermentation products from starch-containing material comprising the steps of:

(a) liquefying starch-containing material in the presence of an alpha-amylase;

(b) saccharifying the liquefied material obtained in step (a) using a carbohydrate-source generating enzyme;

(c) fermenting using a fermenting organism;

wherein a metallo protease is present i) during fermentation, and/or ii) before, during, and/or after liquefaction, and a pullulanase is present i) during fermentation, and/or ii) before, during, and/or after liquefaction.

Saccharification step (b) and fermentation step (c) may be carried out either sequentially or simultaneously. The metallo protease may be added during saccharification and/or fermentation when the process is carried out as a sequential saccharification and fermentation process and before or during fermentation when steps (b) and (c) are carried out simultaneously (SSF process). The metallo protease may also advantageously be added before liquefaction (pre-liquefaction treatment), i.e., before or during step (a), and/or after liquefaction (post liquefaction treatment), i.e., after step (a). The pullulanase is most advantageously added before or during liquefaction, i.e., before or during step (a).

The fermentation product, such as especially ethanol, may optionally be recovered after fermentation, e.g., by distillation. Suitable starch-containing starting materials are listed in the section “Starch-Containing Materials”-section below. Contemplated enzymes are listed in the “Enzymes”-section below. The liquefaction is preferably carried out in the presence of an alpha-amylase, preferably a bacterial alpha-amylase or acid fungal alpha-amylase. The fermenting organism is preferably yeast, preferably a strain of Saccharomyces cerevisiae. Suitable fermenting organisms are listed in the “Fermenting Organisms”-section above.

In a particular embodiment, the process of the invention further comprises, prior to the step (a), the steps of:

x) reducing the particle size of the starch-containing material, preferably by milling;

y) forming a slurry comprising the starch-containing material and water.

The aqueous slurry may contain from 10-55 w/w-% dry solids (DS), preferably 25-45 w/w-% dry solids (DS), more preferably 30-40 w/w-% dry solids (DS) of starch-containing material. The slurry is heated to above the gelatinization temperature and alpha-amylase, preferably bacterial and/or acid fungal alpha-amylase may be added to initiate liquefaction (thinning). The slurry may in an embodiment be jet-cooked to further gelatinize the slurry before being subjected to alpha-amylase in step (a).

Liquefaction may in an embodiment be carried out as a three-step hot slurry process. The slurry is heated to between 60-95° C., preferably 80-85° C., and alpha-amylase is added to initiate liquefaction (thinning). Then the slurry may be jet-cooked at a temperature between 95-140° C., preferably 105-125° C., for about 1-15 minutes, preferably for about 3-10 minutes, especially around about 5 minutes. The slurry is cooled to 60-95° C. and more alpha-amylase is added to finalize hydrolysis (secondary liquefaction). The liquefaction process is usually carried out at pH 4.0-6.5, in particular at a pH from 4.5 to 6.

Saccharification step (b) may be carried out using conditions well-know in the art. For instance, a full saccharification process may last up to from about 24 to about 72 hours, however, it is common only to do a pre-saccharification of typically 40-90 minutes at a temperature between 30-65° C., typically about 60° C., followed by complete saccharification during fermentation in a simultaneous saccharification and fermentation process (SSF process). Saccharification is typically carried out at temperatures from 20-75° C., preferably from 40-70° C., typically around 60° C., and at a pH between 4 and 5, normally at about pH 4.5.

The most widely used process in fermentation product, especially ethanol, production is the simultaneous saccharification and fermentation (SSF) process, in which there is no holding stage for the saccharification, meaning that fermenting organism, such as yeast, and enzyme(s), including the metallo protease, may be added together. SSF may typically be carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours.

### Fermentation Medium

“Fermentation media” or “fermentation medium” refers to the environment in which fermentation is carried out and which includes the fermentation substrate, that is, the carbohydrate source that is metabolized by the fermenting organism.

The fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

### Fermenting Organisms

The term “Fermenting organism” refers to any organism, including bacterial and fungal organisms, suitable for use in a fermentation process and capable of producing the desired fermentation product. Especially suitable fermenting organisms are able to ferment, i.e., convert, sugars, such as glucose or maltose, directly or indirectly into the desired fermentation product. Examples of fermenting organisms include fungal organisms, such as yeast. Preferred yeast includes strains of Saccharomyces spp., in particular, Saccharomyces cerevisiae.

In one embodiment the fermenting organism is added to the fermentation medium so that the viable fermenting organism, such as yeast, count per mL of fermentation medium is in the range from 10⁵ to 10¹², preferably from 10⁷ to 10¹⁰, especially about 5×10⁷.

Commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

### Starch-Containing Materials

Any suitable starch-containing material may be used according to the present invention. The starting material is generally selected based on the desired fermentation product. Examples of starch-containing materials, suitable for use in a process of the invention, include whole grains, corn, wheat, barley, rye, milo, sago, cassava, tapioca, sorghum, rice, peas, beans, or sweet potatoes, or mixtures thereof or starches derived therefrom, or cereals. Contemplated are also waxy and non-waxy types of corn and barley.

The term “granular starch” means raw uncooked starch, i.e., starch in its natural form found in cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called “gelatinization” begins. Granular starch to be processed may be a highly refined starch quality, preferably at least 90%, at least 95%, at least 97% or at least 99.5% pure or it may be a more crude starch-containing materials comprising (e.g., milled) whole grains including non-starch fractions such as germ residues and fibers. The raw material, such as whole grains, may be reduced in particle size, e.g., by milling, in order to open up the structure and allowing for further processing. Two processes are preferred according to the invention: wet and dry milling. In dry milling whole kernels are milled and used. Wet milling gives a good separation of germ and meal (starch granules and protein) and is often applied at locations where the starch hydrolysate is used in production of, e.g., syrups. Both dry and wet milling is well known in the art of starch processing and is equally contemplated for a process of the invention. In an embodiment the particle size is reduced to between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen.

### Fermentation Products

The term “fermentation product” means a product produced by a process including a fermentation step using a fermenting organism. Fermentation products contemplated according to the invention include alcohols (e.g., ethanol, methanol, butanol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferred fermentation processes used include alcohol fermentation processes. The fermentation product, such as ethanol, obtained according to the invention, may preferably be used as fuel. However, in the case of ethanol it may also be used as potable ethanol.

### Recovery

Subsequent to fermentation the fermentation product may be separated from the fermentation medium. The slurry may be distilled to extract the desired fermentation product or the desired fermentation product may be extracted from the fermentation medium by micro or membrane filtration techniques. Alternatively the fermentation product may be recovered by stripping. Methods for recovery are well known in the art.

### Enzymes

Even if not specifically mentioned in context of a process of the invention, it is to be understood that enzyme(s) is(are) used in an effective amount.

### Alpha-Amylase

According to the invention any alpha-amylase may be used, such as of fungal, bacterial or plant origin. In a preferred embodiment the alpha-amylase is an acid alpha-amylase, e.g., acid fungal alpha-amylase or acid bacterial alpha-amylase. The term “acid alpha-amylase” means an alpha-amylase (E.C. 3.2.1.1) which added in an effective amount has activity optimum at a pH in the range of 3 to 7, preferably from 3.5 to 6, or more preferably from 4-5.

### Bacterial Alpha-Amylase

According to the invention a bacterial alpha-amylase is preferably derived from the genus Bacillus.

In a preferred embodiment the Bacillus alpha-amylase is derived from a strain of Bacillus lichenifonnis, Bacillus amyloliquefaciens, Bacillus subtilis or Bacillus stearothermophilus, but may also be derived from other Bacillus sp. Specific examples of contemplated alpha-amylases include the Bacillus licheniformis alpha-amylase shown in SEQ ID NO: 4 in WO 99/19467, the Bacillus amyloliquefaciens alpha-amylase SEQ ID NO: 5 in WO 99/19467 and the Bacillus stearothermophilus alpha-amylase shown in SEQ ID NO: 3 in WO 99/19467 (all sequences hereby incorporated by reference). In an embodiment the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NOS: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or U.S. Pat. No. 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta (181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta (181-182) and further comprise a N193F substitution (also denoted I181*+G182*+N193F) compared to the wild-type BSG alpha-amylase amino acid sequence set forth in SEQ ID NO:3 disclosed in WO 99/19467.

### Bacterial Hybrid Alpha-Amylase

A hybrid alpha-amylase specifically contemplated comprises 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with one or more, especially all, of the following substitution:

G48A+T49I+G107A+H156Y+A181T+N190F+I201F+A209V+Q264S (using the Bacillus licheniformis numbering in SEQ ID NO: 4 of WO 99/19467). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467).

In an embodiment the bacterial alpha-amylase is dosed in an amount of 0.0005-5 KNU per g DS, preferably 0.001-1 KNU per g DS, such as around 0.050 KNU per g DS.

### Fungal Alpha-Amylase

Fungal alpha-amylases include alpha-amylases derived from a strain of the genus Aspergillus, such as, Aspergillus oryzae, Aspergillus niger and Aspergillis kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e. at least 70%, at least 75%, at least 80%, at least 85%, at least 90%, at least 95%, at least 96%, at least 97%, at least 98%, at least 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from Aspergillus niger disclosed as “AMYA_ASPNG” in the Swiss-prot/TeEMBL database under the primary accession no. P56271 and described in WO 89/01969 (Example 3—incorporated by reference). A commercially available acid fungal alpha-amylase derived from Aspergillus niger is SP288 (available from Novozymes A/S, Denmark).

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al. J. Ferment. Bioeng 81:292-298 (1996) “Molecular-cloning and determination of the nucleotide-sequence of a gene encoding an acid-stable alpha-amylase from Aspergillus kawachii”; and further as EMBL: #AB008370.

The fungal alpha-amylase may also be a wild-type enzyme comprising a starch-binding domain (SBD) and an alpha-amylase catalytic domain (i.e., none-hybrid), or a variant thereof. In an embodiment the wild-type alpha-amylase is derived from a strain of Aspergillus kawachii.

### Fungal Hybrid Alpha-Amylase

In a preferred embodiment the fungal acid alpha-amylase is a hybrid alpha-amylase. Preferred examples of fungal hybrid alpha-amylases include the ones disclosed in WO 2005/003311 or U.S. Patent Publication no. 2005/0054071 (Novozymes) or U.S. patent application No. 60/638,614 (Novozymes) which is hereby incorporated by reference. A hybrid alpha-amylase may comprise an alpha-amylase catalytic domain (CD) and a carbohydrate-binding domain/module (CBM), such as a starch binding domain, and optional a linker.

Specific examples of contemplated hybrid alpha-amylases include those disclosed in Table 1 to 5 of the examples in U.S. patent application No. 60/638,614, including Fungamyl variant with catalytic domain JA118 and Athelia rolfsii SBD (SEQ ID NO:100 in U.S. 60/638,614), Rhizomucor pusillus alpha-amylase with Athelia rolfsii AMG linker and SBD (SEQ ID NO:101 in U.S. 60/638,614), Rhizomucor pusillus alpha-amylase with Aspergillus niger glucoamylase linker and SBD (which is disclosed in Table 5 as a combination of amino acid sequences SEQ ID NO:20, SEQ ID NO:72 and SEQ ID NO:96 in U.S. application Ser. No. 11/316,535) or as V039 in Table 5 in WO 2006/069290, and Meripilus giganteus alpha-amylase with Athelia rolfsii glucoamylase linker and SBD (SEQ ID NO:102 in U.S. 60/638,614). Other specifically contemplated hybrid alpha-amylases are any of the ones listed in Tables 3, 4, 5, and 6 in Example 4 in U.S. application Ser. No. 11/316,535 and WO 2006/069290 (hereby incorporated by reference)

Other specific examples of contemplated hybrid alpha-amylases include those disclosed in U.S. Patent Publication no. 2005/0054071, including those disclosed in Table 3 on page 15, such as Aspergillus niger alpha-amylase with Aspergillus kawachii linker and starch binding domain.

Contemplated are also alpha-amylases which exhibit a high identity to any of above mention alpha-amylases, i.e., at least 70%, at least 75%, at least 80%, at least 85%, at least 90%, at least 95%, at least 96%, at least 97%, at least 98%, at least 99% or even 100% identity to the mature enzyme sequences.

An acid alpha-amylases may according to the invention be added in an amount of 0.001 to 10 AFAU/g DS, preferably from 0.01 to 5 AFAU/g DS, especially 0.3 to 2 AFAU/g DS or 0.001 to 1 FAU-F/g DS, preferably 0.01 to 1 FAU-F/g DS.

### Commercial Alpha-Amylase Products

Preferred commercial compositions comprising alpha-amylase include MYCOLASE™ from DSM (Gist Brocades), BAN™, TERMAMYL™ SC, FUNGAMYL™, LIQUOZYME™ X, LIQUOZYME™ SC and SAN™ SUPER, SAN™ EXTRA L (Novozymes A/S) and CLARASE™ L-40,000, DEX-LO™, SPEZYME™ FRED, SPEZYME™ AA, and SPEZYME™ DELTA AA (Genencor Int.), FUELZYME™-LF (Verenium Inc), and the acid fungal alpha-amylase sold under the trade name SP288 (available from Novozymes A/S, Denmark).

### Carbohydrate-Source Generating Enzyme

The term “carbohydrate-source generating enzyme” includes glucoamylase (being glucose generators), beta-amylase and maltogenic amylase (being maltose generators) and also pullulanase and alpha-glucosidase. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated blends are mixtures comprising at least a glucoamylase and an alpha-amylase, especially an acid amylase, even more preferred an acid fungal alpha-amylase. The ratio between glucoamylase activity (AGU) and fungal alpha-amylase activity (FAU-F) (i.e., AGU per FAU-F) may in a preferred embodiment of the invention be between 0.1 and 100 AGU/FAU-F, in particular between 2 and 50 AGU/FAU-F, such as in the range from 10-40 AGU/FAU-F, especially when doing one-step fermentation (Raw Starch Hydrolysis—RSH), i.e., when saccharification and fermentation are carried out simultaneously (i.e. without a liquefaction step).

In a conventional starch-to-ethanol process (i.e., including a liquefaction step (a)) the ratio may preferably be as defined in EP 140,410-B1, especially when saccharification in step (b) and fermentation in step (c) are carried out simultaneously.

### Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135,138), and C. thermohydrosulfuricum (WO 86/01831) and Trametes cingulata, Pachykytospora papyracea; and Leucopaxillus giganteus all disclosed in WO 2006/069289; or Peniophora rufomarginata disclosed in WO2007/124285; or a mixture thereof. Also hybrid glucoamylase are contemplated according to the invention. Examples the hybrid glucoamylases disclosed in WO 2005/045018. Specific examples include the hybrid glucoamylase disclosed in Table 1 and 4 of Example 1 (which hybrids are hereby incorporated by reference).

Contemplated are also glucoamylases which exhibit a high identity to any of above mention glucoamylases, i.e., at least 70%, at least 75%, at least 80%, at least 85%, at least 90%, at least 95%, at least 96%, at least 97%, at least 98%, at least 99% or even 100% identity to the mature enzymes sequences mentioned above.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA and AMG™ E (from Novozymes A/S); OPTIDEX™ 300, GC480, GC417 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.0001-20 AGU/g DS, preferably 0.001-10 AGU/g DS, especially between 0.01-5 AGU/g DS, such as 0.1-2 AGU/g DS.

### Beta-Amylase

A beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is NOVOZYM™ WBA from Novozymes A/S, Denmark and SPEZYME™ BBA 1500 from Genencor Int., USA.

### Maltogenic Amylase

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram DS or 0.05-5 MANU/g DS.

### Pullulanase

Pullulanases (E.C. 3.2.1.41, pullulan 6-glucano-hydrolase), are debranching enzymes characterized by their ability to hydrolyze the alpha-1,6-glycosidic bonds in, for example, amylopectin and pullulan.

Specifically contemplated pullulanases according to the present invention include the pullulanases from Bacillus amyloderamificans disclosed in U.S. Pat. No. 4,560,651 (hereby incorporated by reference), the pullulanase disclosed as SEQ ID NO: 2 in WO 01/151620 (hereby incorporated by reference), the Bacillus deramificans disclosed as SEQ ID NO: 4 in WO 01/151620 (hereby incorporated by reference), and the pullulanase from Bacillus acidopullulyticus disclosed as SEQ ID NO: 6 in WO 01/151620 (hereby incorporated by reference) and also described in FEMS Mic. Let. (1994) 115, 97-106.

Additional pullulanases contemplated according to the present invention included the pullulanases from Pyrococcus woesei, specifically from Pyrococcus woesei DSM No. 3773 disclosed in WO92/02614, and the mature protein sequence disclosed as SEQ ID No: 6 herein.

The pullulanase may according to the invention be added in an effective amount which include the preferred amount of about 0.0001-10 mg enzyme protein per gram DS, preferably 0.0001-0.10 mg enzyme protein per gram DS, more preferably 0.0001-0.010 mg enzyme protein per gram DS. Pullulanase activity may be determined as NPUN. An Assay for determination of NPUN is described in the “Materials & Methods”-section below.

Suitable commercially available pullulanase products include PROMOZYME D, PROMOZYME™ D2 (Novozymes A/S, Denmark), OPTIMAX L-300 (Genencor Int., USA), and AMANO 8 (Amano, Japan).

### Composition Comprising a Metallo Protease, or a Metallo Protease and a Pullulanase

According to this aspect the invention relates to compositions comprising a metallo protease and a carbohydrate-source generating enzyme and an alpha-amylase, preferably glucoamylase, and/or an acid alpha-amylase, or a composition comprising a metallo protease and a pullulanase, and/or a carbohydrate-source generating enzyme and/or an alpha-amylase.

The metallo protease may be any metallo proteases, including the ones listed in the “Metallo protease”-section above. In a preferred embodiment the metallo protease is classified as EC 3.4.24, more preferred EC 3.4.24.39. In a preferred embodiment the metallo protease is derived from a strain of the genus Thermoascus, preferably a strain of Thermoascus aurantiacus, especially Thermoascus aurantiacus CGMCC No. 0670, or a homoglous metallo protease having at least 80% identity to SEQ ID NO: 1, or at least about 82%, or at least about 85%, or at least about 90%, or at least about 95%, or at least about 97%.

The carbohydrate-source generating enzyme may be any carbohydrate-source generating enzyme, including the ones listed in the “Carbohydrate-Source Generating Enzymes”-section above. In a preferred embodiment the carbohydrate-source generating enzyme is a glucoamylase. In an preferred embodiment the glucoamylase is selected from the group derived from a strain of Aspergillus, preferably Aspergillus niger or Aspergillus awamori, a strain of Talaromyces, especially Talaromyces emersonii; or a strain of Athelia, especially Athelia rolfsii; a strain of Trametes, preferably Trametes cingulata; a strain of the genus Pachykytospora, preferably a strain of Pachykytospora papyracea; or a strain of the genus Leucopaxillus, preferably Leucopaxillus giganteus; or a strain of the genus Peniophora, preferably a strain of the species Peniophora rufomarginata; or a mixture thereof.

The alpha-amylase may be any alpha-amylase, including the ones mentioned in the “Alpha-Amylases”-section above. In a preferred embodiment the alpha-amylase is an acid alpha-amylase, especially an acid fungal alpha-amylase. In a preferred embodiment the alpha-amylase is selected from the group of fungal alpha-amylases. In a preferred embodiment the alpha-amylase is derived from the genus Aspergillus, especially a strain of A. niger, A. oryzae, A. awamori, or Aspergillus kawachii, or of the genus Rhizomucor, preferably a strain of Rhizomucor pusillus, or the genus Meripilus, preferably a strain of Meripilus giganteus, or the genus Bacillus, preferably a strain of Bacillus stearothermophilus.

The pullulanase may be any pullulanase, including the ones mentioned in the “Pullulanase” section above. In a one embodiment, the pullulanase is a thermostable pullulanase derived from the genus Pyrococcus, preferably a strain of Pyrococcus woesei.

The compositions may be formulated so that the metallo protease suitably can be used in a process, preferably a process of the invention, in an amount corresponding to 0.0001-10 mg enzyme protein per gram DS, preferably 0.0001-1 mg enzyme protein per gram DS, more preferably 0.0001-0.010 mg enzyme protein per gram DS. The glucoamylase, when present, may be used in an amount of 0.0001-20 AGU per g DS. The acid alpha-amylase, when present, may be used in an amount of 0.001 to 1 FAU-F per g DS. The pullulanase, when present, may be used in an amount of about 0.0001-10 mg enzyme protein per gram DS, preferably 0.0001-0.010 mg enzyme protein per gram DS.

The ratio between glucoamylase activity (AGU) and acid fungal alpha-amylase activity (FAU-F) (i.e., AGU per FAU-F) may in a preferred embodiment of the invention be between 0.1 and 100 AGU/FAU-F, in particular between 2 and 50 AGU/FAU-F, such as in the range from 10-40 AGU/FAU-F glucoamylase and acid alpha-amylase is in the range between 0.3 and 5.0 AFAU/AGU. Above composition of the invention is suitable for use in a process for producing fermentation products, such as ethanol, of the invention.

### Uses

The present invention is also directed to using metallo proteases for producing fermentation products from gelatinized and un-gelatinized starch-containing material, and to using metallo proteases and pullulanases for producing fermentation products from gelatinized starch-containing material.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and de-scribed herein will become apparent to those skilled in the art from the foregoing description. Such modifications are also intended to fall within the scope of the appended claims. In the case of conflict, the present disclosure including definitions will control.

The present invention is described in further detail in the following examples which are offered to illustrate the present invention, but not in any way intended to limit the scope of the invention as claimed. All references cited herein are specifically incorporated by reference for that which is described therein.

### Materials & Methods

### Materials:

Glucoamylase A (AMG A): Glucoamylase derived from Trametes cingulata disclosed in SEQ ID NO: 2 in WO 2006/069289 and available from Novozymes A/S.
Glucoamylase B (AMG B): Glucoamylase derived from Talaromyces emersonii disclosed in SEQ ID No: 7 in WO02/028448 and available from Novozymes A/S.
Alpha-Amylase A (AAA): Hybrid alpha-amylase consisting of Rhizomucor pusillus alpha-amylase with Aspergillus niger glucoamylase linker and SBD disclosed as V039 in Table 5 in WO 2006/069290 (Novozymes A/S).

Alpha-Amylase B (AAB): Alpha amylase derived from Bacillus stearothermophilus as disclosed in WO99/019467 as SEQ ID No: 3 with the double deletion 1181+G182 and substitution N193F, and available from Novozymes A/S.

Alpha-Amylase Z (AAZ): Alpha-amylase as disclosed in Richardson et al. (2002), The Journal of Biological Chemistry, Vol. 277, No 29, Issue 19 July, pp. 267501-26507, referred to as BD5088. This alpha-amylase is the same as the one shown in SEQ ID NO: 4 herein. The mature enzyme sequence starts after the initial “Met” amino acid in position 1. The enzyme is available from Verenium.
Metalloprotease A (MPA): Metallo protease derived from Thermoascus aurantiacus CGMCC No. 0670 disclosed as amino acids 1-177 in SEQ ID NO: 1 herein and amino acids 1-177 in SEQ ID NO: 2 in WO 2003/048353.
Metalloprotease B (MPB): Aminopeptidase 1 derived from Aspergillus oryzae as disclosed as SEQ ID NO: 2 in WO9628542. The mature portion of the enzyme sequence begins at amino acid residue 80 of SEQ ID NO: 2 of WO9628542 and the mature portion of the enzyme is disclosed as SEQ ID NO: 5 herein.
Pullulanase A (PUA): Pullulanase derived from Pyrococcus woesei DSM No. 3773 disclosed in WO92/02614. The mature protein sequence is amino acids 1-1095 of SEQ ID No: 6 herein.
Yeast: RED STAR™ available from Red Star/Lesaffre, USA

### Methods

### Identity

The relatedness between two amino acid sequences or between two nucleotide sequences is described by the parameter “identity”.

For purposes of the present invention the degree of identity between two amino acid sequences, as well as the degree of identity between two nucleotide sequences, may be determined by the program “align” which is a Needleman-Wunsch alignment (i.e. a global alignment). The program is used for alignment of polypeptide, as well as nucleotide sequences. The default scoring matrix BLOSUM50 is used for polypeptide alignments, and the default identity matrix is used for nucleotide alignments. The penalty for the first residue of a gap is −12 for polypeptides and −16 for nucleotides. The penalties for further residues of a gap are −2 for polypeptides, and −4 for nucleotides.

“Align” is part of the FASTA package version v20u6 (see W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA,” Methods in Enzymology 183:63-98). FASTA protein alignments use the Smith-Waterman algorithm with no limitation on gap size (see “Smith-Waterman algorithm”, T. F. Smith and M. S. Waterman (1981) J. Mol. Biol. 147:195-197).

### Protease Assays

AZCL-casein assay

A solution of 0.2% of the blue substrate AZCL-casein is suspended in Borax/NaH₂PO₄ buffer pH9 while stirring. The solution is distributed while stirring to microtiter plate (100 microL to each well), 30 microL enzyme sample is added and the plates are incubated in an Eppendorf Thermomixer for 30 minutes at 45° C. and 600 rpm. Denatured enzyme sample (100° C. boiling for 20 min) is used as a blank. After incubation the reaction is stopped by transferring the microtiter plate onto ice and the coloured solution is separated from the solid by centrifugation at 3000 rpm for 5 minutes at 4° C. 60 microL of supernatant is transferred to a microtiter plate and the absorbance at 595 nm is measured using a BioRad Microplate Reader.

pNA-Assay

50 microL protease-containing sample is added to a microtiter plate and the assay is started by adding 100 microL 1 mM pNA substrate (5 mg dissolved in 100 microL DMSO and further diluted to 10 mL with Borax/NaH₂PO₄ buffer pH9.0). The increase in OD₄₀₅ at room temperature is monitored as a measure of the protease activity.

### Glucoamylase Activity (AGU)

Glucoamylase activity may be measured in Glucoamylase Units (AGU).

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

AMG incubation:

| | |
|---|---|
| Substrate: | maltose 23.2 mM |
| Buffer: | acetate 0.1M |
| pH: | 4.30 ± 0.05 |
| Incubation temperature: | 37° C. ± 1 |
| Reaction time: | 5 minutes |
| Enzyme working range: | 0.5-4.0 AGU/mL |

Color reaction:

| | |
|---|---|
| GlucDH: | 430 U/L |
| Mutarotase: | 9 U/L |
| NAD: | 0.21 mM |
| Buffer: | phosphate 0.12M; 0.15M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum soluble.

A folder EB-SM-0009.02/01 describing this analytical method in more detail is available upon request to Novozymes A/S, Denmark, which folder is hereby included by reference.

### Acid Alpha-Amylase Activity (AFAU)

When used according to the present invention the activity of an acid alpha-amylase may be measured in AFAU (Acid Fungal Alpha-amylase Units). Alternatively, activity of acid alpha-amylase may be measured in AAU (Acid Alpha-amylase Units).

### Acid Alpha-Amylase Units (AAU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

Standard Conditions/Reaction Conditions:

Substrate: Soluble starch. Concentration approx. 20 g DS/L.

Buffer: Citrate, approx. 0.13 M, pH=4.2

Iodine solution: 40.176 g potassium iodide+0.088 g iodine/L

City water 15°-20° dH (German degree hardness)

pH: 4.2

Incubation temperature: 30° C.

Reaction time: 11 minutes

Wavelength: 620 nm

Enzyme concentration: 0.13-0.19 AAU/mL

Enzyme working range: 0.13-0.19 AAU/mL

The starch should be Lintner starch, which is a thin-boiling starch used in the laboratory as colorimetric indicator. Lintner starch is obtained by dilute hydrochloric acid treatment of native starch so that it retains the ability to color blue with iodine. Further details can be found in EP 0140,410 B2, which disclosure is hereby included by reference.

### Determination of FAU-F

FAU-F Fungal Alpha-Amylase Units (Fungamyl) is measured relative to an enzyme standard of a declared strength.

Reaction conditions

| | |
|---|---|
| Temperature | 37° C. |
| pH | 7.15 |
| Wavelength | 405 nm |
| Reaction time | 5 min |
| Measuring time | 2 min |

A folder (EB-SM-0216.02) describing this standard method in more detail is available on request from Novozymes A/S, Denmark, which folder is hereby included by reference.

Acid alpha-amylase activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

$$\begin{matrix}STARCH + IODINE \\ λ = 590 nm \\ blue \text{/} violet\end{matrix} \begin{matrix}{\to }_{40 ° , pH 2 , 5}^{ALPHA \text{-} AMYLASE} \\ t = 23 sec .\end{matrix} \begin{matrix}DEXTRINS + OLIGOSACCHARIDES \\ decoloration\end{matrix}$$

Standard Conditions/Reaction Conditions:
Substrate: Soluble starch, approx. 0.17 g/L
Buffer: Citrate, approx. 0.03 M
Iodine (I2): 0.03 g/L
CaCl2: 1.85 mM
pH: 2.50±0.05
Incubation temperature: 40° C.
Reaction time: 23 seconds
Wavelength: 590 nm
Enzyme concentration: 0.025 AFAU/mL
Enzyme working range: 0.01-0.04 AFAU/mL

A folder EB-SM-0259.02/01 describing this analytical method in more detail is available upon request to Novozymes A/S, Denmark, which folder is hereby included by reference.

### Determination of Pullulanase Activity (NPUN)

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan.

1 ml diluted sample or standard is incubated at 40° C. for 2 minutes. 0.5 ml 2% red pullulan, 0.5 M KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

### EXAMPLES

### Example 1

### Effect of Metallo-Proteases (MPA or MPB) on α-Amylase A (AAA) and Glucoamylase A (AMG A) Combination in Simultaneous Saccharification and Fermentation (SSF) Process

All treatments were evaluated via mini-scale fermentations. 410 g of ground yellow dent corn (with an average particle size around 0.5 mm) was added to 590 g tap water. The mixture was supplemented with 3.0 ml 1 g/L penicillin and 1 g of urea. The pH of the slurry was adjusted to 4.5 with 40% H₂SO₄. Dry solid (DS) level was determined to be 35 wt. %. Approximately 5 g of the slurry was added to 20 ml vials. Each vial was dosed with the amount of enzyme shown in Table 1 and Table 3 below, followed by addition of 200 micro liters yeast propagate/5 g slurry. Vials were incubated at 32° C. Nine replicate fermentations of each treatment were run. Three replicates were selected for 24 hours, 48 hours and 70 hours time point analysis. Vials were vortexed at 24, 48 and 70 hours and analyzed by HPLC. The HPLC preparation consisted of stopping the reaction by addition of 50 micro liters of 40% H₂SO₄, centrifuging, and filtering through a 0.45 micrometer filter. Samples were stored at 4° C. until analysis. Agilent™ 1100 HPLC system coupled with RI detector was used to determine ethanol and oligosaccharides concentration. The separation column was aminex HPX-87H ion exclusion column (300 mm×7.8 mm) from BioRad™. Average ethanol yield (g/L) for each group is summarized in Table 2 and Table 4.

TABLE 1

| | | | AMG A | |
|---|---|---|---|---|
| | | AAA | (AGU/ | MPA |
| Group | Treatments | (FAU-F/gDS) | gDS) | (μg/gDS) |
| 1 | AA 1 + AMG A | 0.0475 | 0.5 | 0 |
| 2 | AAA + AMG A + MPA | 0.0475 | 0.5 | 20 |
| 3 | AAA + AMG A + MPA | 0.0475 | 0.5 | 40 |
| 4 | AAA + AMG A + MPA | 0.0475 | 0.5 | 80 |
| 5 | AAA + AMG A + MPA | 0.0475 | 0.5 | 100 |

TABLE 2

| | Group | | | | |
|---|---|---|---|---|---|
| Time (hr) | 1 | 2 | 3 | 4 | 5 |
| 24 | 110.88 | 111.75 | 110.58 | 114.38 | 114.18 |
| 48 | 148.21 | 150.75 | 152.11 | 153.86 | 153.87 |
| 70 | 158.42 | 159.98 | 160.97 | 161.85 | 162.64 |

TABLE 3

| | | | AMG A | MPA or |
|---|---|---|---|---|
| | | | dose | MPB |
| | | AAA dose | (AGU/ | dose |
| Group | Treatments | (FAU-F/gDS) | gDS) | (μg/gDS) |
| 1a | AAA + AMG A | 0.0475 | 0.5 | 0 |
| 2a | AAA + AMG A + MPB | 0.0475 | 0.5 | 20 |
| 3a | AAA + AMG A + MPA | 0.0475 | 0.5 | 20 |

TABLE 4

| | Group | | |
|---|---|---|---|
| Time (hr) | 1a | 2a | 3a |
| 24 | 101.83 | 106.00 | 108.23 |
| 48 | 137.30 | 143.15 | 148.44 |
| 70 | 144.56 | 149.20 | 154.73 |

### Example 2

Small scale mashes were prepared as follows: about 14 g ground corn, about 12 g backset, and about 13 g water were mixed in a rapid viscoanalyzer cup for a total weight of 40 g. The pH of the corn slurry was adjusted to 5.4. For liquefaction, the enzymes were added to the cup/mixer and placed into the RVA wherein a fixed temperature ramp up to 85° C. with continuous mixing was achieved. The samples were held at 85° C. for 90 minutes with continuous mixing, cooled down and supplemented with 3.0 ml 1 g/L penicillin and 1 g of urea, and further subjected to simultaneous saccharification and fermentation (SSF) with AMG B.

Four small scale mashes were made: 1) control with AAB alone; 2) AAB+PUA (5 μg EP/g DS); 3) AAB+MPA (50 μg EP/g DS) and 4) AAB+PUA+MPA. These mashes were then simultaneously saccharified and fermented (SSF) for 54 hours using AMG B as the glucoamylase. The CO₂ weight loss over time was measured and ethanol quantified using the HPLC after 24 and 54 hours of SSF. For simplification of the data being presented and for purposes of illustration only, the 54 hour HPLC results are summarized below in Table 5.

The addition of the combination of alpha-amylase (AAB), thermostable pullulanase (PUA) and metallo protease (MPA) in liquefaction shows a synergistic effect resulting in a significant benefit in increased ethanol yield (+2.4% relative to control) over the addition of any one of the enzymes alone, or any pair of enzymes at the same concentration.

TABLE 5
54 hours fermentation

| | | Std Dev | EtOH % |
|---|---|---|---|
| Sample | Ethanol g/L | (EtOH) | of control |
| AAB Control | 111.010 | 0.685 | 100.000 |
| AAB + PUA (5 ug) | 109.683 | 0.645 | 98.805 |
| AAB + MPA (50 ug) | 111.144 | 0.242 | 100.121 |
| AAB + PUA (5 ug) + MPA (50 ug) | 113.667 | 0.066 | 102.393 |

### Example 3

Corn mashes were prepared as follows: AAZ (activity of 16.3 KNU(S)/g) was dosed into the whole corn slurry at 0.04% w/w starch dsb (dry solids basis) and held for 30 minutes at 90° C. and at pH 5.4. The slurry was then passed through a lab scale jet cooker at 110° C. with a 10 minute hold time. After the jet cooker, another 0.01% dose of AAZ was added and the liquefied mash held for 90 minutes at 85° C. The final DE of the mash was 13.37. The AAB mash (activity of 240 KNU(S)/g) was made in the same manner as the AAZ mash except for the AAB initial dosage was 0.02% w/w starch dsb, the pH was 5.8, and the second dose of 0.01% AAB was added after the jet cooking step. The final DE of the mash was 13.01.

5, 10, or 50 μg EP/g DS of PUA, MPA, or both were added to the cooled jet-cooked mashes as indicated in Table 6 below, and the mashes were heated back up to 85° C. for 2 hours at pH 5.4 (AAZ) or pH 5.8 (AAB). The treated mashes were then subjected to SSF with AMG B for 54 hours. The ethanol yields were quantified by HPLC. A summary of the results are shown in Table 6.

The combination of thermostable pullulanase (PUA) and metallo protease (MPA) with either AAZ or AAB prepared mashes shows a significant benefit in increased ethanol yield over the addition of any one of the enzymes alone. The benefit was still present even when the MPA dosage was reduced from 50 μg EP/g DS to 10 μg EP/g DS.

TABLE 6
54 hours fermentation

| | | | EtOH % | EtOH % |
|---|---|---|---|---|
| | Ethanol | Std Dev | of AAB | of AAZ |
| Sample | g/L | (EtOH) | control | control |
| AAB | 122.631 | 0.508 | 100.000 | 96.418 |
| AAZ | 127.187 | 0.794 | 103.715 | 100.000 |
| AAZ + MPA (10) + PUA (5) | 133.516 | 0.436 | 108.876 | 104.976 |
| AAZ + MPA (50) + PUA (5) | 135.169 | 1.486 | 110.224 | 106.276 |
| AAZ + PUA (5) | 130.459 | 1.165 | 106.383 | 102.573 |
| AAZ + MPA (50) | 132.338 | 0.651 | 107.915 | 104.050 |
| AAZ + MPA (10) | 128.746 | 1.681 | 104.986 | 101.226 |

1-50. (canceled)

51. A process for producing a fermentation product, comprising simultaneously saccharifying and fermenting a starch-containing material using a carbohydrate-source generating enzyme and a fermenting organism at a temperature below the initial gelatinization temperature of said starch-containing material in the presence of a metalloprotease.

52. The process of claim 51, wherein the metalloprotease is derived from a strain of Thermoascus.

53. The process of claim 52, wherein the metalloprotease is derived from a strain of Thermoascus aurantiacus.

54. The process of claim 51, wherein the metalloprotease has at least 80% sequence identity to the sequence of amino acids 1-177 of SEQ ID NO: 1.

55. The process of claim 51, wherein the metalloprotease has at least 85% sequence identity to the sequence of amino acids 1-177 of SEQ ID NO: 1.

56. The process of claim 51, wherein the metalloprotease has at least 90% sequence identity to the sequence of amino acids 1-177 of SEQ ID NO: 1.

57. The process of claim 51, wherein the metalloprotease has at least 95% sequence identity to the sequence of amino acids 1-177 of SEQ ID NO: 1.

58. The process of claim 51, wherein the metalloprotease has at least 97% sequence identity to the sequence of amino acids 1-177 of SEQ ID NO: 1.

59. The process of claim 51, wherein the metalloprotease comprises the sequence of amino acids 1-177 of SEQ ID NO: 1.

60. The process of claim 51, wherein the starch-containing material is granular starch.

61. The process of claim 51, wherein the starch-containing material is selected from the group consisting of corn, wheat, barley, rye, milo, sago, cassava, tapioca, sorghum, rice and potatoes.

62. The process of claim 51, wherein the temperature is between 25° C. and 40° C.

63. The process of claim 51, wherein the carbohydrate-source generating enzyme is selected from the group consisting of glucoamylase, alpha-glucosidase, maltogenic amylase, and beta-amylase.

64. The process of claim 51, wherein an alpha-amylase is present.

65. The process of claim 51, further comprising recovering the fermentation product.

66. The process of claim 51, wherein the fermentation product is an alcohol, preferably ethanol, especially fuel ethanol, potable ethanol and/or industrial ethanol.

67. The process of claim 66, wherein the fermentation product is ethanol.

68. A process for producing a fermentation product, comprising:
(a) liquefying a starch-containing material in the presence of an alpha-amylase;
(b) saccharifying the liquefied material obtained in step (a) using a carbohydrate-source generating enzyme;
(c) fermenting using a fermenting organism;
wherein a metalloprotease is present i) during fermentation, and/or ii) before, during, and/or after liquefaction.

69. The process of claim 68, wherein the step (b) and (c) are carried out sequentially or simultaneously (i.e., SSF process).

70. A composition comprising a metalloprotease, a carbohydrate-source generating enzyme and an alpha-amylase.


SEQUENCE LISTING (6 sequences)

SEQ ID NO: 1 - protein, 355 aa, Thermoascus aurantiacus
  SIGNAL (1)..(19)
  PROPEP (20)..(178)
  mat_peptide (179)..()
MRLVASLTALVALSVPVFPAAVNVKRASSYLEITLSQVSNTLIKAVVQNTGSDELSFVHLNFFKDPAPVKKVSVYRDGSEVQFEGILSRYKSTGLSRDAFTYLAPGESVEDVFDIASTYDLTSGGPVTIRTEGVVPYATANSTDIAGYISYSSNVLTIDVDGAAAATVSKAITPLDRRTRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 2 - DNA, 1267 bp, Aspergillus oryzae
  CDS (2)..(70)
  mat_peptide (71)..()
  CDS (71)..(1129)
  CDS (1133)..(1195)
  CDS (1199)..(1267)
ccacgcgtccgcggacgcgtgggcgagttcaaaagccatttccagtacttcaagtccttcaagcttcaagatgcgtttcatttctgtctcctctcttcttttagccctggcaccggctctcaatgccgttcctgtagaggttgccggtagtgcccaaggacttgatgtgactcttagccaggtgggaaatactcggatcaaggccgtggtaaagaacactggcagcgaggatgtcacctttgtgcacctcaacttcttcaaggatgccgctccggtgcagaaggtgtctctgttccgcaatgcgaccgaggttcaattccagggtatcaagcagcgtcttatcaccgaaggcttgtccgatgatgctttgacaactcttgcccctggtgctactatcgaggacgagttcgatatcgcaagtactagtgacctgtccgagggtggtaccatcacgatcaacagcaacggtttagtacctattaccaccgataacaaggtcactggatacattccattcacctcgaacgagctctccattgatgtagatgcagctgaggccgcgagtgttactcaagcagttaagatcctggaacgccgcaccaaggtcacttcctgctctggcagcagattgtcggcccttcagactgctctgagaaacacagtctctttggcacgtgcagctgctactgccgcgcagtcgggatcttcctcccgtttccaggagtatttcaagacgacatccagctccacccgtagcacggttgctgctcgcctgaacgccgttgctaacgaggccgcgtcgacctcttcgggaagtaccacgtactactgcagcgacgtgtatggatactgcagctccaacgtgcttgcgtatacccttccgtcttataacatcatcgccaactgcgacctctactattcctatcttccggcactgactagcacctgccatgctcaggaccaggccaccaccaccctgcatgagttcactcacgcccccggtgtgtacagccctggcactgacgaccttggctatggatactcggctgccaccgccttgagtgccagtcaggctctgctgaatgccgacacctatgccttgtttgccaacgctgtcaacctcaactgttagacgcacgtacttgcaatcggcaatttggaaggagtacttcctgcaaggtgtattggaaagctgtagagtgaatatatgattggtgctgatggcattaaagtttatacaatgagatacaatggagccattcacctg

SEQ ID NO: 3 - protein, 420 aa, Aspergillus oryzae
HASADAWASSKAISSTSSPSSFKMRFISVSSLLLALAPALNAVPVEVAGSAQGLDVTLSQVGNTRIKAVVKNTGSEDVTFVHLNFFKDAAPVQKVSLFRNATEVQFQGIKQRLITEGLSDDALTTLAPGATIEDEFDIASTSDLSEGGTITINSNGLVPITTDNKVTGYIPFTSNELSIDVDAAEAASVTQAVKILERRTKVTSCSGSRLSALQTALRNTVSLARAAATAAQSGSSSRFQEYFKTTSSSTRSTVAARLNAVANEAASTSSGSTTYYCSDVYGYCSSNVLAYTLPSYNIIANCDLYYSYLPALTSTCHAQDQATTTLHEFTHAPGVYSPGTDDLGYGYSAATALSASQALLNADTYALFANAVNLNCTHVLAIGNLEGVLPARCIGKLSEYMIGADGIKVYTMRYNGAIHL

SEQ ID NO: 4 - protein, 436 aa, Artificial Sequence
  Synthetic construct
MAKYSELEKGGVIMQAFYWDVPSGGIWWDTIRQKIPEWYDAGISAIWIPPASKGMGGAYSMGYDPYDFFDLGEYDQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASQESYAAYLRSIGIDAWRFDYVKGYAPWVVKDWLNWWGGWAVGEYWDTNVDAVLNWAYSSGAKVFDFALYYKMDEAFDNKNIPALVSALQNGQTVVSRDPFKAVTFVANHDTDIIWNKYPAYAFILTYEGQPTIFYRDYEEWLNKDKLKNLIWIHENLAGGSTDIVYYDNDELIFVRNGYGDKPGLITYINLGSSKAGRWVYVPKFAGACIHEYTGNLGGWVDKYVYSSGWVYLEAPAYDPANGQYGYSVWSYCGVG

SEQ ID NO: 5 - protein, 298 aa, Aspergillus oryzae
YPDSVQHNETVQNLIKSLDKKNFETVLQPFSEFHNRYYKSDNGKKSSEWLQGKIQEIISASGAKGVTVEPFKHSFPQSSLIAKIPGKSDKTIVLGAHQDSINLDSPSEGRAPGADDDGSGVVTILEAFRVLLTDEKVAAGEAPNTVEFHFYAGEEGGLLGSQDIFEQYSQKSRDVKAMLQQDMTGYTKGTTDAGKPESIGIITDNVDENLTKFLKVIVDAYCTIPTVDSKCGYGCSDHASATKYGYPAAFAFESAFGDDSPYIHSADDTIETVNFDHVLQHGKLTLGFAYELAFADSL

SEQ ID NO: 6 - protein, 1095 aa, Pyrococcus woesei
ANNIVKAEEPKPLNVIIVWHQHQPYYYDPVQGIYTRPWVRLHAANNYWKMAHYLTEFPDIHVTIDLSGSLIAQLADYMNGAKDIYQIISEKIANGEPLTYDEKWFMLQAPGGFFDHTIPWNGEPVTDENGNPIRDFWDRYTELKDKMLAAKQKYANLPLEEQKVAVTNEFTEQDYIDLAVLFNLAWIDYNYIISTPELKALYDKVDEGGYTREDLKTVLYHQMWLLNNTFKEHEKINLLLGNGNVEVTVVPYAHPIGPILNDFGWSEDFDAHVKKAHELYKKYLGGGVATPRGGWAAESALNDKTLEILAENGWQWVMTDQMVLERMGIPYSIENYYRPWVAEFNGKKIYLFPRNHDLSDRVGFRYSGMNQYEAVEDFINELLKIQKYNYDGSLVYVITLDGENPWEHYPYDGKLFLETLYKRLSELQEAGLIRTLTPTEYIQLYGDKANKLTPQMMERLDFTTEERVEALKVANSLGELYDLAGVTEEMQWPESSWIDGTLSTWIGEPQENYAWYWLYLARRTLMENKDKMDSASWEKAYEYLLRAEASDWFWWYGNDQDSGQDYSFDRYFKTYLYEIYKLAGVEPPSYLYGNYFPDGAPYTVRALEGLKEGDVKEYSSLSPVAEGVKVFFDSQGLHFIIKGSLDKFEISIYEKDKRIGNTFTLLQKKPDKIRYDVFPFVRDSVGLMITKHIVYKDGKAEIYNATDYEGYEKIGEAQVSVNGDEIEVIVPFEYLETPEDFYFAVSTVDELGMLEVITTPVNLKLPVQVKGVVLVDIADPEGDDHGPGTYTYPTDKVFVEGAFDLLRFRMLEQTDAYVMEFYFKELGGNPWNGPNGFSLQIIEVYLDFKEGGNTSAIKMFPDGPGANVQLDPEHPWDVAFRIAGWDYGNLIVLANGTVYQGEMQISADPTKNAVIVKLPKKYLSIGDYGLYAAVLVGSQDGYGPDKWRPVAVEAEQWKLGGADPQAVVDNLAPRVVDMLVPEGFKPTQEEQLSSYDVEKKELATVYMITLVSGSGEKEEEVEEETPTQTETQTPTETRTETKTPTETTTTTPTETKETPTQTTTTQPARTETQGGICGPGLIVLLAALGVLRRRS